(19) United States
(12) Patent Application Publication
(10) Pub. No.: US 2010/0234846 A1
(43) Pub. Date: Sep. 16, 2010

(54) INTRAMEDULLARY RADIAL HEAD LOCKING PIN IMPLANT

(76) Inventor: W. Andrew Eglseder, Owings Mills, MD (US)

Correspondence Address: WHITEFORD, TAYLOR & PRESTON, LLP;ATTN: GREGORY M STONE, SEVEN SAINT PAUL STREET, BALTIMORE, MD, 21202-1626, US

(21) Appl. No.: 12/722,918

(22) Filed: Mar. 12, 2010

Related U.S. Application Data
(60) Provisional application No. 61/159,901, filed on Mar. 13, 2009.

(51) Int. Cl. A61B 17/56

(52) U.S. Cl. 606/62; 606/96

(57) ABSTRACT

The invention provides an intramedullary radial head locking pin implant device having a substantially cylindrical elongate member with a proximal end and a distal end, the implant narrowing at its distal end. The implant includes at least three openings for locking screws to engage therethrough. One opening is located near the proximal end of the implant for a locking screw to be inserted in the radial head; the two remaining openings are located near the distal end of the implant for two locking screws to be inserted into the bone shaft. The proximal end of the implant is formed as a fixation point for securing an external jig system to align the locking screws.

### CROSS REFERENCE TO RELATED APPLICATION

This application is based upon and claims benefit of copending and co-owned U.S. Provisional Patent Application Ser. No. 61/159,901 entitled “Intramedullary Radial Head Locking Pin Implant”, filed with the U.S. Patent and Trademark Office on Mar. 13, 2009 by the inventor herein, the specification of which is incorporated herein by reference.

### BACKGROUND OF THE INVENTION

1. Field of the Invention

The present invention relates generally the treatment of fractures and to devices used for stabilizing bone fractures, and more particularly the invention relates to apparatus for effecting such treatments as are applicable to fractures of bones such as the radius.

2. Background

The radius is one of two long bones found in the human forearm. The radius, like other bones, is susceptible to a variety of fractures and other dislocations. For example, fractures of the radius are a common result of forward falls, with the palms facing downward. In such falls, force exerted on the hands and wrist at impact frequently produces displacement of one or more bone fragments created distal to the fracture site. Unfortunately, internal fixation of such displaced bone fragments using bone plates has proved problematic.

Fixation may be performed dorsally. In this approach, a surgeon may apply a reducing force on the fracture by attaching a bone plate to the dorsal side of the radius. However, unless the bone plate has a very low profile, dorsal tendons and ligaments overlying the bone plate may rub against it, producing tendon irritation or even tendon rupture.

Despite the potential advantages of dorsal fixation, attachment of the bone plate to the dorsal side of the fractured radius may complicate reduction of the radius. The radius of elderly patients, in particular, generally contains porous bone of poor quality. Accordingly, bone screws inserted into the radius through openings in the bone plate may not achieve enough purchase in the bone to hold bone fragments in position against the bone plate. Moreover, these bone screws may be even less effective at gaining enough purchase to pull bone fragments toward the plate to aid in reduction of the fracture. Thus, there is a need for an alternative strategy for reducing and fixing radial fractures.

Fractures of the radial head can lead to joint pain and elbow instability. For a radial head fracture, it is typical to remove the head of the radius for replacement with a prosthetic radial head. Examples of a radial head prosthesis are shown in U.S. Patent Application Publication Nos. 2003/0212457 and 2001/0037154, and in U.S. Pat. Nos. 6,656,225; 6,361,563; 6,270,529; and 6,217,616.

Alternatively, fractures of long bones are often successfully treated using an intramedullary fixation device in the form of a rod or pin, without a prosthesis. These fixation devices are useful not only with transverse simple fractures, but also with comminuted and segmental fractures. In essence, intramedullary pins stabilize fractures by acting as an internal splint. Early non-fixed rods were not always adequately secured to the bone, particularly with comminuted fractures. In some instances, these non-fixed rods would become loose. A section of the bone could then rotate around the rod or shift axially, causing a rotational displacement about the fracture line, a gap, or other discontinuity. In order to provide rotational stability to the fractured bone, many conventional intramedullary rods now have transverse holes for receiving a fastener such as a screw. The transverse holes are typically located at opposed ends of the rod, which, for convenience, are referred to as the proximal end and the distal end. By securing the rod to the bone using transfixation screws, rotation of the bone relative to the rod is prevented. It is also known that the use of both proximal and distal locking reduces axial displacement of bone along the rod and provides additional torsional rigidity.

Intramedullary rods are inserted in the bone using well known techniques. In the case of the radius, sufficient tissue is removed to allow the rod to be inserted into the intramedullary canal. Various alignment devices are available for aligning the proximal end of the rod so that the surgeon can locate and align the transfixation holes. With respect to the distal end of the rod, however, it is much more difficult to align the transfixation holes. Typically, the holes in the distal end must be located by x-ray imaging using an image intensifier; several attempts may be required before transfixation is successful. Thus, the transfixation of the distal ends of intramedullary rod is usually a difficult procedure.

It will be appreciated by those skilled in the art that the intramedullary canal of the radius does not have a uniform cross section along its entire length. Many prior art intramedullary rods are not well adapted for use in bones having these non-uniform cavities. In addition, the relative placement of the transfixation holes of known prior art intramedullary rods does not readily facilitate the use of a C-arm, X-ray machine to locate holes at the distal end.

An example of a bone fixation device is described in U.S. Pat. No. 5,766,174 to Perry, which discloses an intramedullary rod having a cylindrical portion and a rectangular portion. The rod has fixation holes and provides rotational stability. An alignment device with a drill guide is also disclosed.

Another example of a bone fixation device of the prior art is described in U.S. Pat. No. 6,270,499 to Leu et al., which discloses an intramedullary nail and an intramedullary nail head that can be set and fixed on the proximal end of the intramedullary nail. At least one threaded bore is provided between the ends of the intramedullary nail.

U.S. Pat. No. 6,629,976 to Gnos et al. and U.S. Pat. No. 6,692,496 to Wardlaw describe an intramedullary nail having a fixture for connecting an aiming device to position transverse bores in order to secure the intramedullary nail. The nail may be slightly curved in order to match the axis of the bone.

U.S. Pat. No. 7,153,309 to Huebner et al. discloses a guide system for bone repair devices. The system may include a guide device having a coupling portion and a guide portion to guide a hole-forming tool for aligning predefined holes for fasteners into the bone.

None of the prior art discloses an intramedullary rod for the proximal end of the radius and the radial head. It would be desirable to provide an intramedullary bone fixation device for use with the proximal end of the radius and the radial head.

### SUMMARY OF THE INVENTION

Accordingly, it is an object of the present invention to provide a radial head implant that avoids the disadvantages of the prior art.

It is an object of the present invention to provide a radial head implant that enables fixation of radial head fractures using an intramedullary pin. A related object of the present invention to provide a radial head implant that is held in place by locking screws.

It is further an object of the present invention to provide a radial head implant that allows fixation without external plates and screws.

It is another object of the present invention to provide a radial head implant having an external jig system. A related object of the present invention to provide an external jig system for aligning locking screws with an intramedullary pin.

These and other objects of the present invention are accomplished by providing an intramedullary radial head locking pin implant device having a substantially cylindrical elongate member with a proximal end and a distal end, the implant narrowing at its distal end. The implant includes at least three openings for locking screws to engage therethrough. One opening is located near the proximal end of the implant for a locking screw to be inserted in the radial head; the two remaining openings are located near the distal end of the implant for locking screws to be inserted into the bone shaft. The proximal end of the implant is formed as a fixation point for securing an external jig system to align the locking screws.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1 is a perspective view of an intramedullary locking pin implant according to a first embodiment of the present invention.

### DETAILED DESCRIPTION OF EXEMPLARY EMBODIMENTS

The invention summarized above may be better understood by referring to the following description, which should be read in conjunction with the accompanying drawings. This description of an embodiment, set out below to enable one to build and use an implementation of the invention, is not intended to limit the invention, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

The invention provides a guide system and an intramedullary bone repair device for use with radial head fractures, including a method and apparatus for guiding a hole-forming tool and/or a fastener toward a predefined position(s) of a bone-repair device. The system may be used for coupling the fastener to the bone-repair device, after placement of the fastener through a bone, from an opposing surface of the bone. The bone-repair device may include an intramedullary pin. Accordingly, the guide system may be, for example, a fixation system for fixing a fractured or excised bone, or an implant system for replacing a portion of the bone. The system may include a guide device that connects in a predefined relation to the bone-repair device. The guide device may be configured so that a guide portion of the guide device and the bone-repair device are disposed on generally opposing sides of the bone. The guide device may be configured to correspond to a particular bone-repair device, so that the guide device properly targets hole-forming tools and/or fasteners through the bone and then to predefined position(s) of the bone-repair device.

The guide system may be configured to repair any suitable bone damage, and/or perform any other suitable function, in or on any suitable bone(s). For example, the guide system may be used as a fixation system to reduce and/or fix bone discontinuities, such as fractures of the radius, tibia, fibula, femur, ulna, humerus, among others.

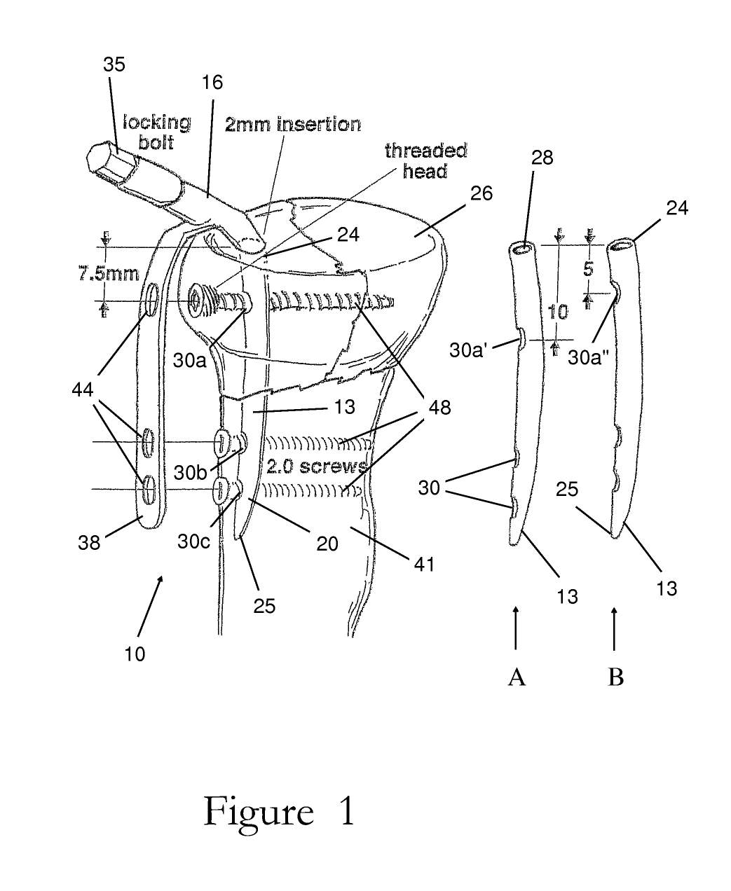

Referring to the drawings, FIG. 1 shows an implant device, indicated generally as 10, according to the present invention. The implant device 10 includes a pin 13 and an alignment jig 16. The pin 13 comprises a substantially cylindrical elongate member 20 defined by a longitudinal axis and having a proximal end 22 and a distal end 25. The pin 13 may be slightly curved along the longitudinal axis with a radius of curvature between approximately 100 and 1000 mm. The elongate member 20 may be narrowed to a blunt point at its distal end 25. The proximal end 22 of the member 20 is formed as a securing head 28 for attaching the alignment jig 16 to the pin 13. The pin 13 has a length and diameter sized and configured for use as an intramedullary implant for fractures of the radial head 26.

The pin 13 has a plurality of openings 30. In a preferred embodiment, at least three openings 30 are provided. At least one opening 30a is located near the proximal end 24 of the pin 13. Other openings 30b, 30c are located near the distal end 25 of the pin 13. In some embodiments, opening 30a may be located approximately 7.5 mm from the proximal end 24 of the pin 13. As shown in the embodiment labeled A, opening 30a′ may be located approximately 10 mm from the proximal end 24 of the pin 13. As shown in the embodiment labeled B, opening 30a″ may be located approximately 5 mm from the proximal end 24 of the pin 13. Other appropriate locations may be used.

The alignment jig 16 has a fixation means that can be removably attached to the securing head 28 of the pin 13 using methods that are well known to one of ordinary skill in the relevant art, such as locking bolt 35. The jig 16 includes a guide tongue 38 for guiding a hole-forming tool and/or a fastener through the bone 41 and then to the pin 13 that is inserted in the bone 41. The guide tongue 38 has a plurality of apertures 44 positioned symmetrically with the openings 30 in the pin 13. The apertures 44 in the guide tongue 38 are guide holes that should match-up with the openings 30 in the pin 13. It is contemplated that an array of possible positions of the guide apertures 44 and the openings 30 may be used. For example, see the different intramedullary pin embodiments at A and B of FIG. 1.

The jig component 16 of the implant device 10 is used as a guide to implant the pin 13, which is intended as an intramedullary device. The jig 16 is removed upon securing the intramedullary pin 13.

In use, the radial head implant device is used for stabilization of radial head fractures using an intramedullary pin with three locking screws 48. The radius is prepared by removing sufficient tissue to allow the pin 13 to be inserted into the intramedullary canal. The pin 13, with the alignment jig 16 attached, is inserted into the intramedullary canal. A drill, or other appropriate device, is used to make holes in the radial head 26 and the shaft of the bone 41. Appropriate fixing devices, such as surgical screws 48 sized and configured for use as described, are inserted transversely of the pin 13, and pass through at least part of the bone 41 to be treated. One screw 48 is inserted into the radial head 26 and two more screws 48 are inserted into the shaft of the bone 41 using the alignment jig 16. The internal nature of the intramedullary pin allows fixation to be performed without external plates and screws, obviating the impact on surrounding soft tissues and ligaments.

The invention provides apparatus for the treatment of a bone fracture, comprising an elongate member 20 adapted for substantially axial insertion into the bone 41 through the radial head 26 such that it spans the fracture; where in one embodiment the elongate member being formed, adjacent its proximal end with a guide jig 16 having fixture location means in the form of a guide tongue 38 adapted to receive a fixing device such as a surgical screw 48 inserted transversely of the elongate member 20 and passing through part at least of the bone 41 to be treated; the line of insertion of the fixing device 48 being defined by the jig 16 temporarily affixable to the securing head 28 at the proximal end 24 of the elongate member 20.

The invention has been described with references to a preferred embodiment. While specific values, relationships, materials and steps have been set forth for purposes of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in the light of the above teachings, those skilled in the art can modify those specifics without departing from the invention taught herein. Having now fully set forth the preferred embodiments and certain modifications of the concept underlying the present invention, various other embodiments as well as certain variations and modifications of the embodiments herein shown and described will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

What is claimed is:

1. An intramedullary pin, comprising:
an elongate pin defined by a longitudinal axis and having a proximal end and a distal end;
a securing head for an alignment jig formed in the proximal end of the pin; and
a plurality of openings formed in the pin,
wherein at least one opening is formed near the proximal end of the pin, and;
at least one additional opening is formed near the distal end of the pin; and
wherein the pin is adapted to be inserted in a radial head.

2. The intramedullary pin according to claim 1, wherein said pin is curved.

3. The intramedullary pin according to claim 2, said pin having a radius of curvature between 100 and 1000 mm.

4. The intramedullary pin according to claim 1, further comprising:
an alignment jig.

5. The intramedullary pin according to claim 4, said alignment jig further comprising:
fixation means that can be removably attached to the securing head.

6. The intramedullary pin according to claim 4, said alignment jig further comprising:
a locking bolt for removably attaching the alignment jig to the pin.

7. The intramedullary pin according to claim 4, said alignment jig further comprising:
a guide tongue.

8. The intramedullary pin according to claim 7, said guide tongue having a plurality of apertures.

9. The intramedullary pin according to claim 8, said plurality of apertures being positioned symmetrically with the openings in said pin.

10. The intramedullary pin according to claim 7, said guide tongue being sized and configured for guiding a hole-forming tool and/or a fastener through a radius bone and then to the pin that is inserted in the bone.

11. A method of stabilizing a radial head fracture, comprising:
providing an elongate pin defined by a longitudinal axis and having a proximal end and a distal end;
said pin comprising a securing head for an alignment jig formed in the proximal end of the pin; and
a plurality of openings formed in the pin,
wherein at least one opening is formed near the proximal end of the pin, and;
at least one additional opening is formed near the distal end of the pin;
providing an alignment jig removably attached to said pin;
preparing the radial head for intramedullar insertion of the pin;
forming a plurality of holes in the radius; and
anchoring the pin inside the medullary canal of the radius.

12. The method according to claim 11, said alignment jig further comprising:
a guide tongue.

13. The method according to claim 12, said guide tongue having a plurality of apertures.

14. The method according to claim 13, said plurality of apertures being positioned symmetrically with the openings in said pin.

15. The method according to claim 12, said guide tongue being sized and configured for guiding a hole-forming tool and/or a fastener through a radius bone and then to the pin that is inserted in the bone.

16. The method according to claim 11, wherein said pin is curved.

17. The method according to claim 16, said pin having a radius of curvature between 100 and 1000 mm.
